(19) United States
(12) Patent Application Publication
(10) Pub. No.: US 2021/0366168 A1
(43) Pub. Date: Nov. 25, 2021

(54) PET IMAGE RECONSTRUCTION METHOD, COMPUTER STORAGE MEDIUM, AND COMPUTER DEVICE

(71) Applicant: SHENZHEN INSTITUTES OF ADVANCED TECHNOLOGY, Nanshan Shenzhen (CN)

(72) Inventors: Zhanli Hu, Nanshan Shenzhen (CN); Yongfeng Yang, Nanshan Shenzhen (CN); Dong Liang, Nanshan Shenzhen (CN); Xin Liu, Nanshan Shenzhen (CN); Hairong Zheng, Nanshan Shenzhen (CN)

(21) Appl. No.: 17/287,040

(22) PCT Filed: Jan. 15, 2019
(86) PCT No.: PCT/CN2019/071746

§ 371 Date: Apr. 20, 2021

(30) Foreign Application Priority Data
Dec. 14, 2018 (CN) 201811536162.1

(51) Int. Cl. G06T 11/00; G06T 5/00; G06T 7/11; A61B 6/03

(52) U.S. Cl. CPC G06T 11/006; G06T 5/002; G06T 7/11; G06T 2207/30004; G06T 2207/10104; G06T 2207/20081; G06T 2207/20021; A61B 6/037

(57) ABSTRACT

The present invention discloses a PET image reconstruction method, a computer storage medium, and a computer device. The method includes: step 1, obtaining projection data Y and a system matrix P of a PET image; step 2, constructing an imaging model equation Y=PX, in which X is a reconstructed PET image; step 3, obtaining the initial reconstructed image X, and iteratively updating the initial reconstructed image X according to a first objective function to obtain a first reconstructed image; step 4, iteratively updating the first reconstructed image according to the second objective function to obtain the second reconstructed image; and step 5, determining whether an iteration condition is satisfied, if yes, outputting the current round of iteration to obtain the second reconstructed image as a final PET reconstructed image, and if not, returning to step 3 and using the second reconstructed image in the current round of iteration as an initial reconstructed image in the next round of iteration. The reconstruction algorithm of the present invention does not depend on a conformity degree between anatomical structure information and functional information, and can distinguish image edges well regardless of whether there is noise interfering with the image edges.

### TECHNICAL FIELD

The present invention relates to the field of information technology, particularly to a PET image reconstruction method, a computer storage medium, and a computer device.

### BACKGROUND ART

The Positron Emission Tomography (PET), i.e., positron tomography imaging, is imaged by injecting a radiotracer into a patient first and then measuring the distribution of radioisotopes in the patient. A PET reconstruction algorithm is mainly divided into two categories including an analytic reconstruction algorithm and an iterative reconstruction algorithm. The analytic reconstruction algorithm mainly includes back projection, filtered back-projection, and Fourier reconstruction, in which the most widely used algorithm is filtered back-projection (FBP). The FBP method is based on a Radon transform. However, the FBP method neither takes into account the spatiotemporal inhomogeneity of system response, nor the noise of the instrument during measurement, resulting in that a reconstructed image contains a large amount of noise. The iterative reconstruction algorithm includes algebraic reconstruction and statistical reconstruction, in which the algebraic reconstruction includes an algebra reconstruction technique (ART) and some new algorithms obtained by further ART-based expansion. The maximum likelihood-expectation maximization (ML-EM) method in statistical reconstruction is currently widely used in clinical practice since ML-EM has a better performance in lesion detection than traditional algorithms, but this method has degraded image quality with the increase of the number of iterations and may generate “checkerboard artifacts”. An early termination of iteration and an integration of penalty terms or some priori knowledge in a likelihood function overcomes the problem of the ML-EM method to some extent.

In brief, the existing PET reconstruction method has the following problems: 1) since only a single-pixel difference is used to distinguish a true edge and noise fluctuation in image reconstruction affected by noise, the reconstructed image does not retain a correct edge; 2) since there is no proper balance between removing noise and retaining detailed information, the reconstructed image loses much detailed information; 3) for under-sampled images and noise images, the lack of good constraint conditions may cause the loss of details to generate blocky artifacts.

### SUMMARY OF THE INVENTION

### (1) Technical Problem to be Solved

The technical problem to be solved by the present invention is how to obtain a PET image with better reconstruction effect.

### (2) Technical Solution

To achieve the above purposes, the present invention adopts the following technical solutions:

A PET image reconstruction method includes the following steps:

step 1, obtaining projection data Y and system matrix P of a PET image;

step 2, constructing an imaging model equation Y=PX, in which X is a reconstructed PET image.

step 3, obtaining the initial reconstructed image X, and iteratively updating the initial reconstructed image X according to a first objective function to obtain a first reconstructed image, in which the first objective function is:

$${X}^{n + 1} = {arg max}_{X \geq 0} {Q}_{L} ( X \text{;} {X}^{n} ) - β {Q}_{U}^{b} ( X , {X}^{n} )$$

in which Q_{L} (X; Xⁿ) is a likelihood surrogate function constructed based on a Poisson random distribution variable, Q_{U}^{b}(X; Xⁿ) is a penalty surrogate function constructed based on neighborhood block priori, Xⁿ is a reconstructed image obtained after an n-th iteration, and β is a regularization parameter;

step 4, iteratively updating the first reconstructed image according to a second objective function to obtain a second reconstructed image, in which the second objective function is a function that is constructed based on dictionary learning; and

step 5, determining whether an iteration condition is satisfied, if yes, outputting the current round of iteration to obtain the second reconstructed image as a final PET reconstructed image, and if not, returning to step 3 and using the second reconstructed image in the current round of iteration as an initial reconstructed image in the next round of iteration.

Alternatively, an expression of the likelihood surrogate function is:

$${Q}_{L} ( X ; {X}_{n} ) = {\sum }_{{ }^{j = 1}}^{{n}_{j}} {p}_{j} ( {\hat{X}}_{j , EM}^{n + 1} log {X}_{j} - {X}_{j} )$$

in which

$${\hat{X}}_{j , EM}^{n + 1} = \frac{{X}_{j}^{n}}{{p}_{j}} {\sum }_{i = 1}^{{n}_{i}} {p}_{ij} \frac{{y}_{1}}{{\overline{y}}_{i}^{n}} , \\ {p}_{j} = {\sum }_{i = 1}^{{n}_{i}} {p}_{ij} , \\ {\overline{y}}^{n} = {PX}^{n} + r ,$$

nⱼ represents a total amount of pixels, pᵢⱼ represents a probability that a j-th pixel is detected by an i-th detector, nᵢ represents a total number of detectors, pⱼ represents a total probability value that the j-th pixel is detected by the nᵢ-th detector, Xⱼ represents a value of the j-th pixel of the reconstructed image X, Xⱼⁿ represents a value of the j-th pixel of the reconstructed image Xⁿ after an n-th iteration, yᵢ represents the projection data detected by the i-th detector, Y̅ᵢⁿ represents the expected projection data, and \ddot{X}_{j,EM}ⁿ⁺¹ represents a value of the j-th pixel of an expectation maximization image.

Alternatively, an expression of the penalty surrogate function is:

$${Q}_{U}^{b} ( X ; {X}_{n} ) = \frac{1}{2} {\sum }_{{ }^{j = 1}}^{{n}_{j}} {{ω}_{j}^{n} ( {X}_{j} - {\hat{X}}_{j , Reg}^{n + 1} )}^{2}$$

in which

$${\hat{X}}_{j , Reg}^{n + 1} = \frac{1}{2 {w}_{j}^{n}} {\sum }_{k ∈ {N}_{j}} {w}_{jk} ( {X}^{n} ) ( {X}_{k}^{n} + {X}_{j}^{n} ) , \\ {w}_{j}^{n} = {\sum }_{k ∈ {N}_{j}} {w}_{jk} ( {X}^{n} ) , \\ {w}_{jk} ( {X}^{n} ) = {\sum }_{l = 1}^{{n}_{j}} {h}_{l} {w}_{jl , kl}^{φ} ( {X}^{n} ) ,$$

ωⱼⁿ represents a weight of the j-th pixel, wⱼₖ represents a weight of the reconstructed image Xⁿ between the j-th pixel and a k-th pixel, \hat{X}_{j,Reg}ⁿ⁺¹ represents a value of the j-th pixel of an intermediate image, Nⱼ represents a neighborhood block centered on the j-th pixel, xₖⁿ represents a value of the k-th pixel of the reconstructed image Xⁿ in the neighborhood block of the j-th pixel after the n-th iteration, jₗ is an l-th pixel in the neighborhood block fⱼ(X), kₗ is the l-th pixel in the neighborhood block fₖ(X), and hₗ is a positive weight vector.

Alternatively, a method of iteratively updating an initial reconstructed image X according to the first objective function to obtain the first reconstructed image includes the steps of:

obtaining an expectation maximization image according to the initial reconstructed image X, a projection data Y and a system matrix P;

performing an image smoothing process on the initial reconstructed image X to obtain an intermediate image; and

generating the first reconstructed image according to the expectation maximization image and the intermediate image.

Alternatively, an expression of the second objective function is:

$${min}_{D , α} {\sum }_{i , j} {‖ {R}_{ij} X - D {α}_{ij} ‖}_{2}^{2} , \\ st . {‖ {α}_{ij} ‖}_{0} \leq {T}_{0} , \text{∀} i , j ,$$

in which X represents the first reconstructed image obtained by reconstruction in step 3, Rᵢⱼ is an operation of obtaining image blocks from X, D is a dictionary based on the image blocks, αᵢⱼ is a sparse representation of Xᵢⱼ with respect to the dictionary D, and T₀ represents a sparsity level to be achieved.

Alternatively, a method of iteratively updating the first reconstructed image according to the second objective function to obtain the second reconstructed image includes the steps of:

segmenting the first reconstructed image to generate a plurality of image blocks;

generating a sparse coefficient for each image block according to each of the image blocks, and pre-trained low-resolution dictionary and high-resolution dictionary;

generating a high-resolution image corresponding to the first reconstructed image according to the sparse coefficient of each image block and the high-resolution dictionary; and

generating and outputting the second reconstructed image according to the first reconstructed image, the high-resolution image, a predetermined fuzzy matrix, and a predetermined down-sampling matrix.

Alternatively, the method of generating a sparse coefficient for each image block according to each of the image blocks and pre-trained low-resolution dictionary and high-resolution dictionary includes the steps of:

constructing a first coefficient constraint condition according to the image blocks, the low-resolution dictionary, a predetermined feature extraction function, and a predetermined first threshold;

constructing a second coefficient constraint condition according to the image blocks, an overlapping area of the image block with a previous image block, the high-resolution dictionary, and a predetermined second threshold; and

calculating the sparse coefficient of the image block that satisfies the first coefficient constraint condition and the second coefficient constraint condition according to a predetermined coefficient calculation formula.

Alternatively, the reconstruction method prior to image segmentation of the first reconstructed image further includes the steps of:

randomly initializing the low-resolution dictionary and the high-resolution dictionary; and

performing a joint training on the low-resolution dictionary and the high-resolution dictionary according to a predetermined low-resolution PET training image set, a predetermined high-resolution PET training image set, a size of an image block in the low-resolution PET training image set, and a size of an image block in the high-resolution PET image set.

The present invention also discloses a computer storage medium that stores a PET image reconstruction program, and the above PET image reconstruction method is realized when the PET image reconstruction program is executed by a processor.

The present invention also discloses a computer device, including a memory, a processor, a PET image reconstruction program stored in the memory, in which any of the above PET image reconstruction methods is realized when the PET image reconstruction program is executed by the processor.

### (3) Beneficial Effects

The PET image reconstruction method disclosed by the present invention overcomes the problem of containing a large amount of noise in the reconstructed image by adding a Poisson random noise variable in the reconstruction process, performs image reconstruction through neighborhood block priori, overcomes the problem in the prior art that the reconstructed image does not retain a correct edge by using a single-pixel difference to distinguish a true edge and noise fluctuation, and removes image noise and artifacts by further performing an update iteration on the first reconstructed image based on dictionary learning. Therefore, the reconstruction algorithm of the present invention does not depend on a conformity degree between anatomical structure information and functional information, and can distinguish image edges well regardless of whether there is noise interfering with the image edges.

### BRIEF DESCRIPTION OF THE DRAWINGS

FIG. 1 is a flow diagram of a PET image reconstruction method according to an embodiment of the present invention;

FIG. 2 is a flow diagram of a method of iteratively updating an initial reconstructed image X according to a first objective function to obtain a first reconstructed image in accordance with the embodiment of the present invention;

FIG. 3 is a flow diagram of a method of iteratively updating the first reconstructed image according to a second objective function to obtain a second reconstructed image in accordance with the embodiment of the present invention; and

FIGS. 4a to 4d are PET images obtained by different reconstruction methods respectively.

### DETAILED DESCRIPTION

To provide a clearer understanding of the purpose, technical solutions and advantages of the present invention, the present invention will be further described in detail with reference to the accompanying drawings and embodiments. It should be understood that the specific embodiments described herein are for illustration of the present invention only and are not intended to limit the present invention.

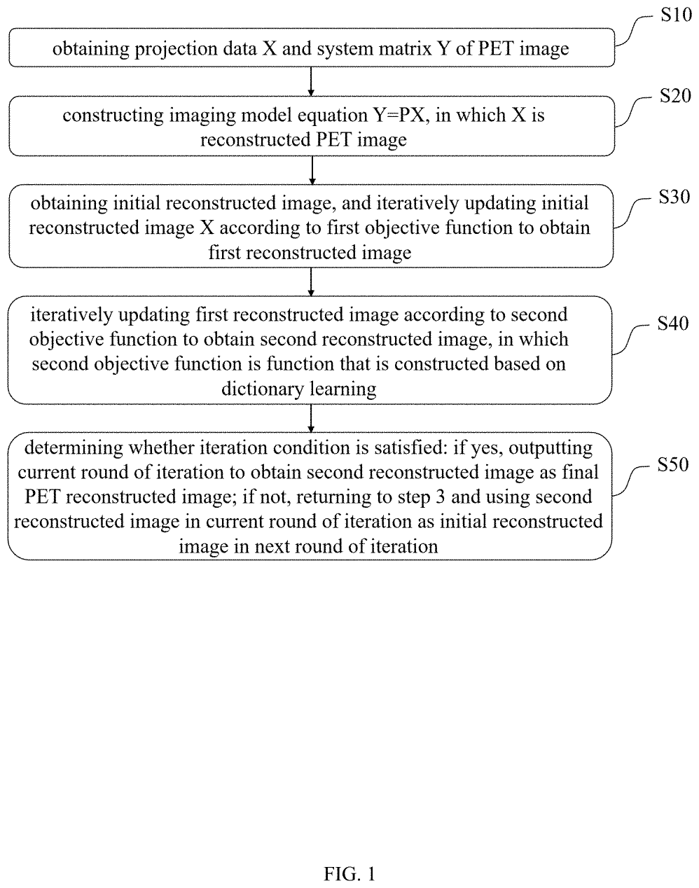

As shown in FIG. 1, a PET image reconstruction method according to an embodiment of the present invention includes the following steps.

Step 1: S10, obtaining projection data Y and system matrix P of a PET image.

On the one hand, from the perspective of simulation experiment, the projection data of the PET image can be simulated data, that is, an existing system matrix can be obtained by using simulated projection data corresponding to an existing simulated PET image; on the other hand, from the perspective of actual measurement, the projection data can be obtained by scanning through a PET scanning system, and then a system matrix intrinsic to a PET system is calculated according to geometric structural information of the PET scanning system.

Step 2: S20, constructing an imaging model equation Y=PX, in which X is a reconstructed PET image.

Step 3: S30, obtaining an initial reconstructed image X, and iteratively updating the initial reconstructed image X according to the first objective function to obtain the first reconstructed image, in which the first objective function is:

$${X}^{n + 1} = {arg max}_{X \geq 0} {Q}_{L} ( X \text{;} {X}^{n} ) - β {Q}_{U}^{b} ( X , {X}^{n} ) ,$$

in which Q_{L}(X; Xⁿ) is a likelihood surrogate function constructed based on a Poisson random distribution variable, Q_{U}^{b}(X; Xⁿ) is a penalty surrogate function constructed based on neighborhood block priori, Xⁿ is a reconstructed image obtained after an n-th iteration, and β is a regularization parameter.

Specifically, setting a maximum number of iterations as Maxlter=100, a regularization parameter as β=2⁻⁷, and a hyper-parameter as δ=1e⁻⁹ to initialize parameters. The initial reconstructed image X is given a value of Xⱼ¹=1 to initialize the image, in which j represents a j-th pixel.

Further, an expression of the likelihood surrogate function is:

$${Q}_{L} ( X ; {X}_{n} ) = {\sum }_{{ }^{j = 1}}^{{n}_{j}} {p}_{j} ( {\hat{X}}_{j , EM}^{n + 1} log {X}_{j} - {X}_{j} )$$

in which

$${\hat{X}}_{j , EM}^{n + 1} = \frac{{X}_{j}^{n}}{{p}_{j}} {\sum }_{i = 1}^{{n}_{i}} {p}_{ij} \frac{{y}_{1}}{{\overline{y}}_{i}^{n}} , \\ {p}_{j} = {\sum }_{i = 1}^{{n}_{i}} {p}_{ij} , \\ {\overline{y}}^{n} = {PX}^{n} + r ,$$

nⱼ represents a total amount of pixels, pᵢⱼ represents a probability that the j-th pixel is detected by an i-th detector, nᵢ represents a total number of detectors, pⱼ represents a total probability value that the j-th pixel is detected by an n-th detector, Xⱼ represents a value of the j-th pixel of the reconstructed image X, Xⱼⁿ represents a value of the j-th pixel of the reconstructed image Xⁿ after the n-th iteration, yᵢ represents the projection data detected by the i-th detector, y̅ᵢⁿ represents the expected projection data that is obtained by the initial reconstructed image X through affine transformation at each iteration, and \hat{X}_{j,EM}ⁿ⁺¹ represents a value of the j-th pixel of an expectation maximization image.

Further, an expression of the penalty surrogate function is:

$${Q}_{U}^{b} ( X ; {X}_{n} ) = \frac{1}{2} {\sum }_{{ }^{j = 1}}^{{n}_{j}} {{ω}_{j}^{n} ( {X}_{j} - {\hat{X}}_{j , Reg}^{n + 1} )}^{2}$$

in which

$${\hat{X}}_{j , Reg}^{n + 1} = \frac{1}{2 {w}_{j}^{n}} {\sum }_{k ∈ {N}_{j}} {w}_{jk} ( {X}^{n} ) ( {X}_{k}^{n} + {X}_{j}^{n} ) , \\ {w}_{j}^{n} = {\sum }_{k ∈ {N}_{j}} {w}_{jk} ( {X}^{n} ) , \\ {w}_{jk} ( {X}^{n} ) = {\sum }_{l = 1}^{{n}_{j}} {h}_{l} {w}_{jl , kl}^{φ} ( {X}^{n} ) ,$$

ωⱼⁿ represents a weight of the j-th pixel, wⱼₖ represents a weight of the reconstructed image Xⁿ between the j-th pixel and a k-th pixel, \hat{X}_{j,Reg}ⁿ⁺¹ represents a value of the j-th pixel of a smooth image, Nⱼ represents a neighborhood block centered on the j-th pixel, Xₖⁿ represents a value of the k-th pixel of the reconstructed image Xⁿ in the neighborhood block of the j-th pixel after the n-th iteration, jₗ is an l-th pixel in the neighborhood block fⱼ(X), kₗ is the l-th pixel in the neighborhood block fₖ(x), and hₗ is a positive weight vector.

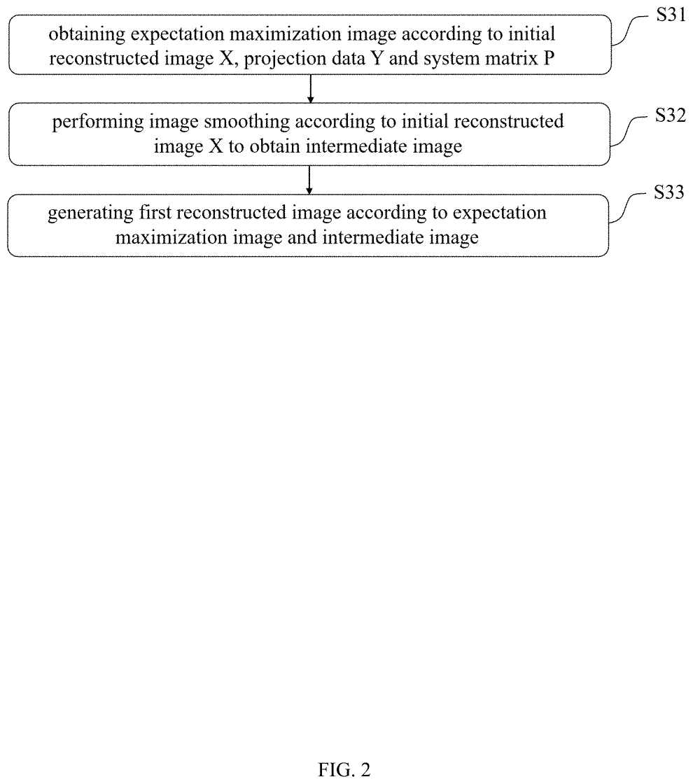

Further, as shown in FIG. 2, step 3 specifically includes the following steps.

Step S31: obtaining the expectation maximization image according to the initial reconstructed image X, a projection data Y and a system matrix P.

Specifically, for the j-th pixel, an EM image is firstly updated through sinogram {yᵢ}, i.e., each pixel is updated according to an objective function

$${\hat{X}}_{j , EM}^{n + 1} = \frac{{X}_{j}^{n}}{{p}_{j}} {\sum }_{i = 1}^{{n}_{i}} {p}_{ij} \frac{{y}_{1}}{{\overline{y}}_{i}^{n}}$$

to finally obtain the expectation maximization image.

Step S32: performing image smoothing according to the initial reconstructed image X to obtain an intermediate image.

Specifically, for the j-th pixel, each pixel is updated according to an objective function

$${\hat{X}}_{j , Reg}^{n + 1} = \frac{1}{2 {w}_{j}^{n}} {\sum }_{k ∈ {N}_{j}} {w}_{jk} ( {X}^{n} ) ( {X}_{k}^{n} + {X}_{j}^{n} )$$

to finally obtain the intermediate image.

Step S33: generating the first reconstructed image according to the expectation maximization image and the intermediate image.

Specifically, for the j-th pixel, pixel-wise segmentation is performed according

$${X}_{j}^{n + 1} = \frac{2 {\hat{x}}_{j , EM}^{n + 1}}{\sqrt{{( 1 - {β}_{j}^{n} {\hat{X}}_{j , Reg}^{n + 1} )}^{2} + 4 {β}_{j}^{n}} {\hat{X}}_{j , EM}^{n + 1} + ( 1 - {β}_{j}^{n} {\hat{X}}_{j , Reg}^{n + 1} )}$$

to an objective function to finally obtain the first

$${β}_{j}^{n} = \frac{β {w}_{j}^{n}}{{p}_{j}} .$$

reconstructed image, in which

Step 4: S40, iteratively updating the first reconstructed image according to a second objective function to obtain a second reconstructed image, in which the second objective function is a function that is constructed based on dictionary learning.

Specifically, the expression of the second objective function is:

$${min}_{D , α} {\sum }_{i , j} {‖ {R}_{ij} X - D {α}_{ij} ‖}_{2}^{2} , \\ st . {‖ {α}_{ij} ‖}_{0} \leq {T}_{0} , \text{∀} i , j ,$$

in which X represents the first reconstructed image obtained by reconstruction in step 3, Rᵢⱼ is an operation of obtaining image blocks from X, D is a dictionary based on the image blocks, αᵢⱼ is a sparse representation of Xᵢⱼ with respect to the dictionary D, and T₀ represents a sparsity level to be achieved.

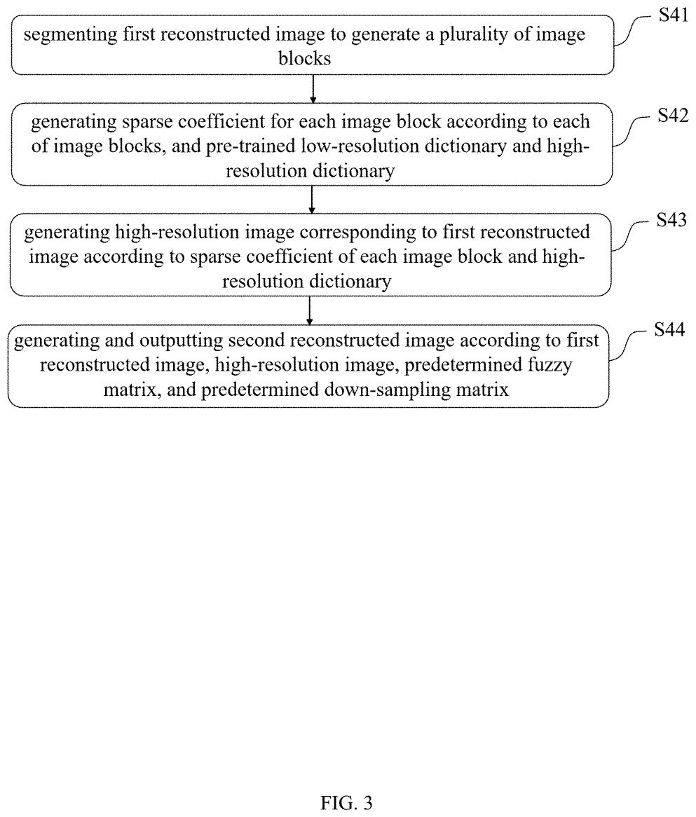

Further, as shown in FIG. 3, step S40 includes the following steps.

Step S41: segmenting the first reconstructed image to generate a plurality of image blocks.

Specifically, the first reconstructed image can be segmented by a predetermined image segmentation algorithm to generate a plurality of image blocks of the first reconstructed image, in which each image block has the same size during segmentation, and an overlapping area exists between the front and back adjacent image blocks.

Step S42: generating a sparse coefficient for each image block according to each of the image blocks, and pre-trained low-resolution dictionary and high-resolution dictionary.

Specifically, step S42 includes the following steps:

constructing a first coefficient constraint condition according to the image blocks, the low-resolution dictionary, a predetermined feature extraction function, and a predetermined first threshold;

constructing a second coefficient constraint condition according to the image blocks, an overlapping area of the image block with a previous image block, the high-resolution dictionary, and a predetermined second threshold; and

calculating the sparse coefficient of the image block that satisfies the first coefficient constraint condition and the second coefficient constraint condition according to a predetermined coefficient calculation formula.

Step S43: generating a high-resolution image corresponding to the first reconstructed image according to the sparse coefficient of each image block and the high-resolution dictionary; and

step S44: generating and outputting the second reconstructed image according to the first reconstructed image, the high-resolution image, a predetermined fuzzy matrix, and a predetermined down-sampling matrix. In this way, one iteration process is completed.

Further, the reconstruction method prior to image segmentation of the first reconstructed image also includes the steps of:

randomly initializing the low-resolution dictionary and the high-resolution dictionary; and

performing a joint training on the low-resolution dictionary and the high-resolution dictionary according to a predetermined low-resolution PET training image set, a predetermined high-resolution PET training image set, a size of an image block in the low-resolution PET training image set, and a size of an image block in the high-resolution PET image set.

Step 5: S50, determining whether an iteration condition is satisfied, if yes, outputting the current round of iteration to obtain the second reconstructed image as a final PET reconstructed image, and if not, returning to step 3 and using the second reconstructed image in the current round of iteration as an initial reconstructed image in the next round of iteration; for this embodiment, the iteration condition is that the number of iterations reaches the maximum number of iteration Maxlter=100.

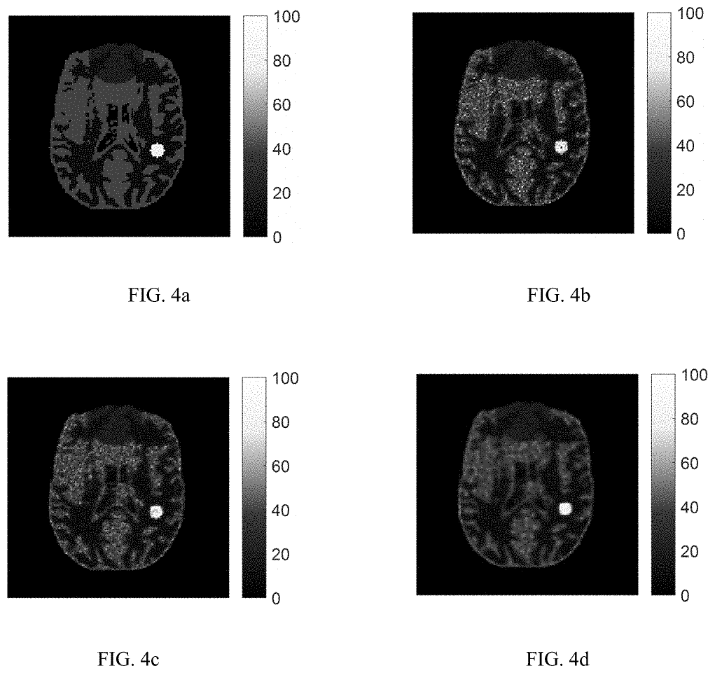

The following is a comparison of images obtained by using a reconstruction method in the prior art and the reconstruction method of the present invention. FIG. 4a is an original simulated PET emission image as a comparison image in this embodiment, FIG. 4b is a PET reconstruction image obtained based on single-pixel regularization, FIG. 4c is a PET reconstruction image obtained based on neighborhood block regularization, and FIG. 4d is a PET reconstruction image obtained by using the reconstruction method of the present invention. As can be seen from FIGS. 4b and 4c, although edges and tumor regions of the image can be reconstructed based on the method of single-pixel and neighborhood block regularization, the reconstructed image contains a large amount of noise, the edge of tumor is fuzzy, and the tumor region contains artifacts. As shown in FIG. 4d, the image reconstructed by using the method of the present invention inhibits noise and artifacts, and the edges and some detailed information of the tumor region are also well retained.

The PET image reconstruction method disclosed by the present invention overcomes the problem of containing a large amount of noise in the reconstructed image by adding a Poisson random noise variable in the reconstruction process, performs image reconstruction through neighborhood block priori, overcomes the problem in the prior art that the reconstructed image does not retain a correct edge by using a single-pixel difference to distinguish a true edge and noise fluctuation, and removes image noise and artifacts by further performing an update iteration on the first reconstructed image based on dictionary learning. Therefore, the reconstruction algorithm of the present invention does not depend on a conformity degree between anatomical structure information and functional information, and can distinguish image edges well regardless of whether there is noise interfering with the image edges.

Further, embodiments of the present invention also disclose a computer storage medium that stores a PET image reconstruction program, and the above PET image reconstruction method is realized when the PET image reconstruction program is executed by a processor.

Further, embodiments of the present invention also discloses a computer device, including a memory, a processor, a PET image reconstruction program stored in the memory, in which any of the above PET image reconstruction methods is realized when the PET image reconstruction program is executed by the processor.

Specific embodiments of the present invention have been described above. Although some embodiments have been shown and described, it should be understood by those skilled in the art that modifications and improvements can be made to these embodiments without departing from the principles and spirits of the present invention as defined by claims and the equivalents thereof, and these modifications and improvements are also within the scope of the present invention.

1. A PET image reconstruction method, comprising:
step 1, obtaining projection data Y and a system matrix P of a PET image;
step 2, constructing an imaging model equation Y=PX, wherein X is a reconstructed PET image.
step 3, obtaining the initial reconstructed image X, and iteratively updating the initial reconstructed image X according to a first objective function to obtain a first reconstructed image, wherein the first objective function is:
$${X}^{n + 1} = {arg max}_{X \geq 0} {Q}_{L} ( X \text{;} {X}^{n} ) - β {Q}_{U}^{b} ( X , {X}^{n} )$$
wherein Q_{L}(X; Xⁿ) is a likelihood surrogate function constructed based on a Poisson random distribution variable, Q_{U}^{b}(X; Xⁿ) is a penalty surrogate function constructed based on neighborhood block priori, Xⁿ is a reconstructed image obtained after an n-th iteration, and β is a regularization parameter;
step 4, iteratively updating the first reconstructed image according to a second objective function to obtain a second reconstructed image, wherein the second objective function is a function that is constructed based on dictionary learning; and
step 5, determining whether an iteration condition is satisfied, if yes, outputting the current round of iteration to obtain the second reconstructed image as a final PET reconstructed image, and if not, returning to step 3 and using the second reconstructed image in the current round of iteration as an initial reconstructed image in the next round of iteration.

2. The PET image reconstruction method according to claim 1,
wherein an expression of the likelihood surrogate function is:
$${Q}_{L} ( X \text{;} {X}_{n} ) = {\sum }_{{ }^{j = 1}}^{{n}_{j}} {p}_{j} ( {\hat{X}}_{j , EM}^{n + 1} log {X}_{j} - {X}_{j} )$$
wherein
$${\hat{X}}_{j , EM}^{n + 1} = \frac{{X}_{j}^{n}}{{p}_{j}} {\sum }_{j = 1}^{{n}_{j}} {p}_{ij} \frac{{y}_{i}}{{\overline{y}}_{i}^{n}} , \\ {p}_{j} = {\sum }_{i = 1}^{{n}_{i}} {p}_{ij} , \\ {\overline{y}}^{n} = {PX}^{n} + r ,$$
nⱼ represents a total amount of pixels, pᵢⱼ represents a probability that a j-th pixel is detected by an i-th detector, nᵢ represents a total number of detectors, pⱼ represents a total probability value that the j-th pixel is detected by nᵢ detectors, Xⱼ represents a value of the j-th pixel of the reconstructed image X, Xⱼⁿ represents a value of the j-th pixel of the reconstructed image Xⁿ after the n-th iteration, yᵢ represents the projection data detected by the i-th detector, represents expected projection data, and \hat{X}j,EMⁿ⁺¹ represents a value of the j-th pixel of an expectation maximization image.

3. The PET image reconstruction method according to claim 2,
wherein an expression of the penalty surrogate function is:
$${Q}_{U}^{b} ( X \text{;} {X}_{n} ) = \frac{1}{2} {\sum }_{{ }^{j = 1}}^{{n}_{j}} {{ω}_{j}^{n} ( {X}_{j} - {\hat{X}}_{j , Reg}^{n + 1} )}^{2}$$
wherein
$${\hat{X}}_{j , Reg}^{n + 1} = \frac{1}{2 {w}_{j}^{n}} {\sum }_{k ∈ {N}_{j}} {w}_{jk} ( {X}^{n} ) ( {X}_{k}^{n} + {X}_{j}^{n} ) , \\ {w}_{j}^{n} = {\sum }_{k ∈ {N}_{j}} {w}_{jk} ( {X}^{n} ) , \\ {w}_{jk} ( {X}^{n} ) = {\sum }_{l = 1}^{{n}_{l}} {h}_{l} {w}_{jl , kl}^{φ} ( {X}^{n} ) ,$$
ωⱼⁿ represents a weight of the j-th pixel, wⱼₖ represents a weight of the reconstructed image Xⁿ between the j-th pixel and a k-th pixel, \hat{X}_{j,Reg}ⁿ⁺¹ represents a value of the j-th pixel of an intermediate image, Nⱼ represents a neighborhood block centered on the j-th pixel, Xₖⁿ represents a value of the k-th pixel of the reconstructed image Xⁿ in the neighborhood block of the j-th pixel after the n-th iteration, jₗ is an l-th pixel in the neighborhood block fⱼ(X), kₗ is the l-th pixel in the neighborhood block fₖ(X), and hₗ is a positive weight vector.

4. The PET image reconstruction method according to claim 1,
wherein the method of iteratively updating the initial reconstructed image X according to the first objective function to obtain the first reconstructed image includes the steps of:
obtaining an expectation maximization image according to the initial reconstructed image X, a projection data Y and a system matrix P;
performing an image smoothing process on the initial reconstructed image X to obtain an intermediate image; and
generating the first reconstructed image according to the expectation maximization image and the intermediate image.

5. The PET image reconstruction method according to claim 1,
wherein an expression of the second objective function is:
$${min}_{D , α} {\sum }_{i , j} {‖ {R}_{ij} X - D {α}_{ij} ‖}_{2}^{2} , \\ st . {‖ {α}_{ij} ‖}_{0} \leq {T}_{0} , \text{∀} i , j ,$$
wherein X represents the first reconstructed image obtained by reconstruction in step 3, Rᵢⱼ is an operation of obtaining image blocks from X, D is a dictionary based on the image blocks, αᵢⱼ is a sparse representation of Xᵢⱼ with respect to the dictionary D, and T₀ represents a sparsity level to be achieved.

6. The PET image reconstruction method according to claim 1,
wherein the method of iteratively updating the first reconstructed image according to the second objective function to obtain the second reconstructed image includes the steps of:
segmenting the first reconstructed image to generate a plurality of image blocks;
generating a sparse coefficient for each image block according to each of the image blocks, and pre-trained low-resolution dictionary and high-resolution dictionary;
generating a high-resolution image corresponding to the first reconstructed image according to the sparse coefficient of each image block and the high-resolution dictionary; and
generating and outputting the second reconstructed image according to the first reconstructed image, the high-resolution image, a predetermined fuzzy matrix, and a predetermined down-sampling matrix.

7. The PET image reconstruction method according to claim 6,
wherein the method of generating a sparse coefficient for each image block according to each of the image blocks and pre-trained low-resolution dictionary and high-resolution dictionary includes the steps of:
constructing a first coefficient constraint condition according to the image blocks, the low-resolution dictionary, a predetermined feature extraction function, and a predetermined first threshold;
constructing a second coefficient constraint condition according to the image blocks, an overlapping area of the image block with a previous image block, the high-resolution dictionary, and a predetermined second threshold; and
calculating the sparse coefficient of the image block that satisfies the first coefficient constraint condition and the second coefficient constraint condition according to a predetermined coefficient calculation formula.

8. The PET image reconstruction method according to claim 6,
wherein the reconstruction method prior to image segmentation of the first reconstructed image also includes the steps of:
randomly initializing the low-resolution dictionary and the high-resolution dictionary; and
performing a joint training on the low-resolution dictionary and the high-resolution dictionary according to a predetermined low-resolution PET training image set, a predetermined high-resolution PET training image set, a size of an image block in the low-resolution PET training image set, and a size of an image block in the high-resolution PET image set.

9. A computer storage medium,
wherein a PET image reconstruction program is stored, and the PET image reconstruction method according to claim 1 is realized when the PET image reconstruction program is executed by a processor.

10. A computer device, comprising:
a memory;
a processor; and
a PET image reconstruction program stored in the memory,
wherein a PET image reconstruction method is realized when the PET image reconstruction program is executed by a processor, the PET image reconstruction method including:
step 1, obtaining projection data Y and a system matrix P of a PET image;
step 2, constructing an imaging model equation Y=PX, wherein X is a reconstructed PET image;
step 3, obtaining the initial reconstructed image X, and iteratively updating the initial reconstructed image X according to a first objective function to obtain a first reconstructed image, wherein the first objective function is:
$${X}^{n + 1} = {arg max}_{X \geq 0} {Q}_{L} ( X \text{;} {X}^{n} ) - β {Q}_{U}^{b} ( X , {X}^{n} )$$
wherein Q_{L}(X; Xⁿ) is a likelihood surrogate function constructed based on a Poisson random distribution variable, Q_{U}^{b}(X; Xⁿ) is a penalty surrogate function constructed based on neighborhood block priori, Xⁿ is a reconstructed image obtained after an n-th iteration, and β is a regularization parameter;
step 4, iteratively updating the first reconstructed image according to a second objective function to obtain a second reconstructed image, wherein the second objective function is a function that is constructed based on dictionary learning; and
step 5, determining whether an iteration condition is satisfied, if yes, outputting the current round of iteration to obtain the second reconstructed image as a final PET reconstructed image, and if not, returning to step 3 and using the second reconstructed image in the current round of iteration as an initial reconstructed image in the next round of iteration.

11. The computer device according to claim 10,
wherein an expression of the likelihood surrogate function is:
$${Q}_{L} ( X \text{;} {X}_{n} ) = {\sum }_{{ }^{j = 1}}^{{n}_{j}} {p}_{j} ( {\hat{X}}_{j , EM}^{n + 1} log {X}_{j} - {X}_{j} )$$
wherein
$${\hat{X}}_{j , EM}^{n + 1} = \frac{{X}_{j}^{n}}{{p}_{j}} {\sum }_{j = 1}^{{n}_{j}} {p}_{ij} \frac{{y}_{i}}{{\overline{y}}_{i}^{n}} , \\ {p}_{j} = {\sum }_{i = 1}^{{n}_{i}} {p}_{ij} , \\ {\overline{y}}^{n} = {PX}^{n} + r ,$$
nⱼ represents a total amount of pixels, pᵢⱼ represents a probability that a j-th pixel is detected by an i-th detector, nᵢ represents a total number of detectors, pⱼ represents a total probability value that the j-th pixel is detected by nᵢ detectors, Xⱼ represents a value of the j-th pixel of the reconstructed image X, Xⱼⁿ represents a value of the j-th pixel of the reconstructed image Xⁿ after the n-th iteration, yᵢ represents the projection data detected by the i-th detector, y̅ᵢⁿ represents expected projection data, and \hat{X}_{j,EM}ⁿ⁺¹ represents a value of the j-th pixel of an expectation maximization image.

12. The computer device according to claim 11,
wherein an expression of the penalty surrogate function is:
$${Q}_{U}^{b} ( X \text{;} {X}_{n} ) = \frac{1}{2} {\sum }_{{ }^{j = 1}}^{{n}_{j}} {{ω}_{j}^{n} ( {X}_{j} - {\hat{X}}_{j , Reg}^{n + 1} )}^{2}$$
wherein
$${\hat{X}}_{j , Reg}^{n + 1} = \frac{1}{2 {w}_{j}^{n}} {\sum }_{k ∈ {N}_{j}} {w}_{jk} ( {X}^{n} ) ( {X}_{k}^{n} + {X}_{j}^{n} ) , \\ {w}_{j}^{n} = {\sum }_{k ∈ {N}_{j}} {w}_{jk} ( {X}^{n} ) , \\ {w}_{jk} ( {X}^{n} ) = {\sum }_{l = 1}^{{n}_{l}} {h}_{l} {w}_{jl , kl}^{φ} ( {X}^{n} ) ,$$
ωⱼⁿ represents a weight of the j-th pixel, ωⱼₖ represents a weight of the reconstructed image Xⁿ between the j-th pixel and a k-th pixel, \hat{X}_{j,Reg}ⁿ⁺¹ represents a value of the j-th pixel of an intermediate image, Nⱼ represents a neighborhood block centered on the j-th pixel, Xₖⁿ represents a value of the k-th pixel of the reconstructed image Xⁿ in the neighborhood block of the j-th pixel after the n-th iteration, jₗ is an l-th pixel in the neighborhood block fⱼ(X), kₗ s the l-th pixel in the neighborhood block fₖ(X), and hₗ is a positive weight vector.

13. The computer device according to claim 10,
wherein the method of iteratively updating the initial reconstructed image X according to the first objective function to obtain the first reconstructed image includes the steps of:
obtaining an expectation maximization image according to the initial reconstructed image X, a projection data Y and a system matrix P;
performing an image smoothing process on the initial reconstructed image X to obtain an intermediate image; and
generating the first reconstructed image according to the expectation maximization image and the intermediate image.

14. The computer device according to claim 10,
wherein an expression of the second objective function is:
$${min}_{D , α} {\sum }_{i , j} {‖ {R}_{ij} X - D {α}_{ij} ‖}_{2}^{2} , \\ st . {‖ {α}_{ij} ‖}_{0} \leq {T}_{0} , \text{∀} i , j ,$$
wherein X represents the first reconstructed image obtained by reconstruction in step 3, Rᵢⱼ is an operation of obtaining image blocks from X, D is a dictionary based on the image blocks, αᵢⱼ is a sparse representation of Xᵢⱼ with respect to the dictionary D, and T₀ represents a sparsity level to be achieved.

15. The computer device according to claim 10,
wherein the method of iteratively updating the first reconstructed image according to the second objective function to obtain the second reconstructed image includes the steps of:
segmenting the first reconstructed image to generate a plurality of image blocks;
generating a sparse coefficient for each image block according to each of the image blocks, and pre-trained low-resolution dictionary and high-resolution dictionary;
generating a high-resolution image corresponding to the first reconstructed image according to the sparse coefficient of each image block and the high-resolution dictionary; and
generating and outputting the second reconstructed image according to the first reconstructed image, the high-resolution image, a predetermined fuzzy matrix, and a predetermined down-sampling matrix.

16. The computer device according to claim 15,
wherein the method of generating a sparse coefficient for each image block according to each of the image blocks and pre-trained low-resolution dictionary and high-resolution dictionary includes the steps of:
constructing a first coefficient constraint condition according to the image blocks, the low-resolution dictionary, a predetermined feature extraction function, and a predetermined first threshold;
constructing a second coefficient constraint condition according to the image blocks, an overlapping area of the image block with a previous image block, the high-resolution dictionary, and a predetermined second threshold; and
calculating the sparse coefficient of the image block that satisfies the first coefficient constraint condition and the second coefficient constraint condition according to a predetermined coefficient calculation formula.

17. The computer device according to claim 15,
wherein the reconstruction method prior to image segmentation of the first reconstructed image also includes the steps of:
randomly initializing the low-resolution dictionary and the high-resolution dictionary; and
performing a joint training on the low-resolution dictionary and the high-resolution dictionary according to a predetermined low-resolution PET training image set, a predetermined high-resolution PET training image set, a size of an image block in the low-resolution PET training image set, and a size of an image block in the high-resolution PET image set.
